(19) United States
(12) Patent Application Publication
(10) Pub. No.: US 2026/0335675 A1
(43) Pub. Date: Oct. 29, 2026

(54) SYSTEM AND METHOD FOR MAGNETIC RESONANCE IMAGING-BASED BRAIN TISSUE CLASSIFICATION

(71) Applicants: GE Precision Healthcare LLC, Waukesha, WI (US); The Government of the United States, as Represented by the Director of the Defense Health Agency, Fort Detrick, MD (US); The Brigham and Women’s Hospital, Inc., Boston, MA (US); Uniformed Services University Of The Health Sciences, Bethesda, MD (US)

(72) Inventors: Ante Zhu, Schenectady, NY (US); Hua Li, Sussex, WI (US); Jerome Joseph Maller, Melbourne (AU); James Kevin DeMarco, Bethesda, MD (US); Herman Douglas Morris, Germantown, MD (US); Robert Youngsing Shih, Washington, DC (US); Raymond Yi-Kun Huang, Brookline, MA (US); Alexandra Jacqueline Golby, Boston, MA (US); David Miles Meredith, Canton, MA (US)

(21) Appl. No.: 19/187,540

(22) Filed: Apr. 23, 2025

(51) Int. Cl. A61N 5/10; G01R 33/56; G01R 33/561; G01R 33/563; G06T 12/30; G06V 10/74; G06V 10/764; G06V 20/70

(52) U.S. Cl. CPC A61N 5/1039; G01R 33/5602; G01R 33/5608; G01R 33/5616; G01R 33/56341; G06T 12/30; G06V 10/74; G06V 10/764; G06V 20/70; G06V 2201/031

(57) ABSTRACT

A method includes acquiring a first and a second set of DTI data utilizing a PGSE pulse sequence and an OGSE pulse sequence, respectively; and reconstructing a first FA map, a first RD map, and a first AD map from the first set of DTI data and reconstructing a second FA map, a second RD map, and a second AD map from the second set of DTI data. The method includes reconstructing a first ratio map based on the ratio of the first FA map to the second FA map, a second ratio map based on the ratio of the second RD map to the first RD map, and a third ratio map based on the ratio of the second AD map to the first AD map; and utilizing the first FA map and either the first ratio map or the second ratio map to classify tissue of the brain.

### STATEMENT REGARDING FEDERALLY SPONSORED RESEARCH & DEVELOPMENT

This invention was made with US Government support under contract number W81XWH-16-2-0054 awarded by the US Army Medical Research Acquisition Activity. The Government has certain rights in the invention.

### BACKGROUND

The subject matter disclosed herein relates to medical imaging and, more particularly, to a system and a method for magnetic resonance imaging (MRI)-based brain tissue classification.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During MRI, when a substance such as human tissue is subjected to a uniform magnetic field (polarizing field B₀), the individual magnetic moments of the spins in the tissue partially align with this polarizing field, but precess about it at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, will be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

With regard to treatment of brain tumors, clinical standard of care requires biopsy and histopathology to determine tissue types. However, brain biopsy is invasive and carries appreciable risk. It also has sampling errors, which fails to evaluate spatially tumor heterogeneity. The lack of accurate tissue characterization causes uncertainty that affects clinical decision making, patient outcome, and quality of life. Clinical brain imaging, including standard-of-the-care MRI and computed tomography (CT), provide high sensitivity to lesions allowing radiologists to identify them. The challenge is to differentiate tissue types within and around different lesions.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for classifying brain tissue is provided. The computer-implemented method includes acquiring, via a processing system including one or more processors, a first set of diffusion tensor imaging data having a long diffusion time of a brain of a subject with a magnetic resonance imaging (MRI) scanner utilizing a pulsed gradient spin echo pulse sequence. The computer-implemented method also includes acquiring, via the processing system, a second set of diffusion tensor imaging data having a short diffusion time that is shorter than the long diffusion time of the brain of the subject with the MRI scanner utilizing an oscillating gradient spin echo pulse sequence. The computer-implemented method further includes reconstructing, via the processing system, a first fractional anisotropy map, a first radial diffusivity map, and a first axial diffusivity map from the first set of diffusion tensor imaging data having the long diffusion time. The computer-implemented method even further includes reconstructing, via the processing system, a second fractional anisotropy map, a second radial diffusivity map, and a second axial diffusivity map from the second set of diffusion tensor imaging data having the short diffusion time. The computer-implemented method also includes reconstructing, via the processing system, a first ratio map based on the ratio of the first fractional anisotropy map to the second fractional anisotropy map, a second ratio map based on the ratio of the second radial diffusivity map to the first radial diffusivity map, and a third ratio map based on the ratio of the second axial diffusivity map to the first axial diffusivity map. The computer-implemented method further includes utilizing, via the processing system, the first fractional anisotropy map and either the first ratio map or the second ratio map to classify tissue of the brain.

In another embodiment, a system for classifying brain tissue is provided. The system includes a memory encoding processor-executable routines. The system also includes a processing system including one or more processors and configured to access the memory and to execute the processor-executable routines, wherein the processor-executable routines, when executed by the processing system, cause the processing system to perform actions. The actions include acquiring a first set of diffusion tensor imaging data having a long diffusion time of a brain of a subject with a magnetic resonance imaging (MRI) scanner utilizing a pulsed gradient spin echo pulse sequence. The actions also include acquiring a second set of diffusion tensor imaging data having a short diffusion time that is shorter than the long diffusion time of the brain of the subject with the MRI scanner utilizing an oscillating gradient spin echo pulse sequence. The actions further include reconstructing a first fractional anisotropy map, a first radial diffusivity map, and a first axial diffusivity map from the first set of diffusion tensor imaging data having the long diffusion time. The actions even further include reconstructing a second fractional anisotropy map, a second radial diffusivity map, and a second axial diffusivity map from the second set of diffusion tensor imaging data having the short diffusion time. The actions also include reconstructing a first ratio map based on the ratio of the first fractional anisotropy map to the second fractional anisotropy map, a second ratio map based on the ratio of the second radial diffusivity map to the first radial diffusivity map, and a third ratio map based on the ratio of the second axial diffusivity map to the first axial diffusivity map. The actions further include utilizing the first fractional anisotropy map and either the first ratio map or the second ratio map to classify tissue of the brain.

In a further embodiment, a non-transitory computer-readable medium is provided. The non-transitory computer-readable medium includes processor-executable code that when executed by a processing system including one or more processors, causes the processing system to perform actions. The actions include acquiring a first set of diffusion tensor imaging data having a long diffusion time of a brain of a subject with a magnetic resonance imaging (MRI) scanner utilizing a pulsed gradient spin echo pulse sequence. The actions also include acquiring a second set of diffusion tensor imaging data having a short diffusion time that is shorter than the long diffusion time of the brain of the subject with the MRI scanner utilizing an oscillating gradient spin echo pulse sequence. The actions further include reconstructing a first fractional anisotropy map, a first radial diffusivity map, and a first axial diffusivity map from the first set of diffusion tensor imaging data having the long diffusion time. The actions even further include reconstructing a second fractional anisotropy map, a second radial diffusivity map, and a second axial diffusivity map from the second set of diffusion tensor imaging data having the short diffusion time. The actions also include reconstructing a first ratio map on the ratio of the first fractional anisotropy map to the second fractional anisotropy map, a second ratio map based on the ratio of the second radial diffusivity map to the first radial diffusivity map, and a third ratio map based on the ratio of the second axial diffusivity map to the first axial diffusivity map. The actions further include utilizing the first fractional anisotropy map and either the first ratio map or the second ratio map to classify tissue of the brain.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIGS. 1A-1C depict schematic diagrams of axons in human healthy white matter, solid tumor in malignant brain cancers including adult gliomas, and a mix of axons and tumor cells;

FIGS. 2A-2C depict schematic diagrams of water molecular movements in axons in human healthy white matter, solid tumor in malignant brain cancers including adult gliomas, and a mix of axons and tumor cells;

FIGS. 3A and 3B depict graphs representing simulation of diffusivity and fractional anisotropy, respectively, as a function of diffusion time or frequency for different tissues;

FIGS. 4A and 4B depict graphs representing diffusivity and fractional anisotropy, respectively, in an infiltrative tumor and healthy white matter;

FIG. 5 illustrates an embodiment of a magnetic resonance imaging (MRI) system suitable for use with the disclosed technique, in accordance with aspects of the present disclosure;

FIG. 6 depicts a schematic diagram of a workflow for MRI-based brain tissue classification, in accordance with aspects of the present disclosure;

FIGS. 7A and 7B depicts diffusion tensor imaging MRI pulse sequences, in accordance with aspects of the present disclosure;

FIG. 8 depicts a table summarizing time-dependent diffusivity and fractional anisotropy of different tissue types, in accordance with aspects of the present disclosure;

FIG. 9 illustrates a flow diagram of a method for MRI-based brain tissue classification, in accordance with aspects of the present disclosure, in accordance with aspects of the present disclosure;

FIG. 10 illustrates a flow diagram of a method for the tissue classification step of the method in FIG. 9, in accordance with aspects of the present disclosure;

FIG. 11 depicts images of a brain of a subject comparing tissue classification between the disclosed technique and current techniques, in accordance with aspects of the present disclosure;

FIG. 12 depicts images for illustrating treatment planning using different maps (from the different tissue classification techniques in FIG. 11) for guidance, in accordance with aspects of the present disclosure;

FIG. 13 illustrates the utilization of brain tissue classification utilizing the disclosed technique in tractography, in accordance with aspects of the present disclosure; and

FIG. 14 illustrates the utilization of brain tissue classification utilizing the disclosed technique in dosage delivery in radiation therapy, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

In glioma (the most common malignant brain tumor that occur in both adult and pediatric patients), lesions in the brain present different pathologies including solid tumor, mixtures of tumor and non-tumoral tissues (commonly referred to as infiltrative tumor), necrosis, and mixtures of fluid and non-tumoral tissues (commonly referred to as vasogenic edema). In addition, other brain regions present normal appearing non-tumoral tissues including healthy white matter and healthy gray matter. Differentiating solid tumor, its infiltration, and non-tumoral regions is key to treatment planning. For example, neurosurgeons may resect the solid tumor if there are no eloquent structures (e.g. preserved tracts) within the tumor mass. Radiation therapy can target an infiltrative tumor more accurately if these regions can be accurately identified. Differentiating solid tumor, its infiltration, and non-tumoral regions is also key to post-treatment evaluation.

Clinical standard MRI for brain tumor imaging includes T2-weighted anatomical imaging, fluid attenuated inversion recovery (FLAIR) T2-weighted anatomical imaging, T1-weighted contrast-enhanced anatomical imaging, and diffusion weighted imaging (DWI). Advanced MRI techniques include perfusion MRI, MR spectroscopy (MRS), and amide proton transfer (APT)/chemical exchange saturation transfer (CEST). Lesions can already be identified by radiologists from these image contrasts. However, they have yet to achieve high accuracy in brain tissue classification in oncology. For example, enhancing lesions are identified as solid tumor and necrotic tissues. Non-enhancing T2 signal abnormality tissues are identified as either the infiltrative tumor or benign lesions (vasogenic edema), which have not been deterministically differentiable using current medical imaging techniques.

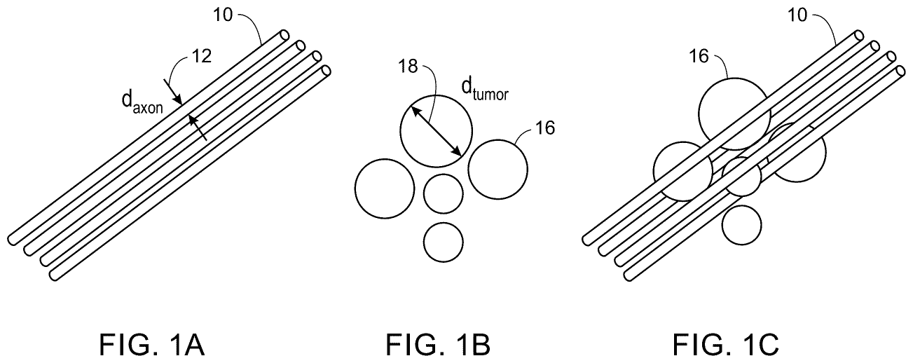

Tumoral lesions and non-tumoral lesions present different cytoarchitectures at the microscopic level. For example, in healthy white matter, axon fibers 10 are the dominant tissues. Their shapes are approximated as cylinders as depicted in FIG. 1A. The axonal diameter 12 (dₐₓₒₙ), i.e., the size of the cylindrical fiber, is relatively small, e.g., radius less than or equal to 2 micrometers. In solid tumor of gliomas, tumor cells 16 are the dominant tissues. They are characterized with spherical shape as depicted in FIG. 1B. The size of tumor cells 16 is relatively large, e.g., radius greater than or equal to 4 micrometers (2 times of the radius is diameter 18 (dₜᵤₘₒᵣ) in the treatment-naïve glioma. Infiltrative tumors are composed of a mixture of axon fibers 10 and tumor cells 16 as depicted in FIG. 1C. Therefore, an imaging method that can differentiate cell shape, alignment, and cell, can classify tissue type in different lesions.

Diffusion tensor imaging (DTI) probes water molecular movement along a certain diffusion encoding direction in the medium. Therefore, DTI has been used for probing cell shape and alignment. Based on Einstein's Equation, the diffusion coefficient (D) is represented by the following equation,

$$D = \frac{< {L}^{2} >}{2 \cdot Δ} , \qquad ( 1 )$$

where L is diffusion length. Fractional anisotropy (FA) describes the anisotropy of the three-dimensional diffusion process in the medium. FA is represented as follows:

$$FA = \sqrt{\frac{1}{2} \cdot \frac{{( {λ}_{1} - {λ}_{2} )}^{2} + {( {λ}_{1} - {λ}_{3} )}^{2} + {( {λ}_{2} - {λ}_{3} )}^{2}}{{λ}_{1}^{2} + {λ}_{2}^{2} + {λ}_{3}^{2}}} , \qquad ( 2 )$$

where λ₁, λ₂, λ₃ are the eigenvalues of the diffusion tensor.

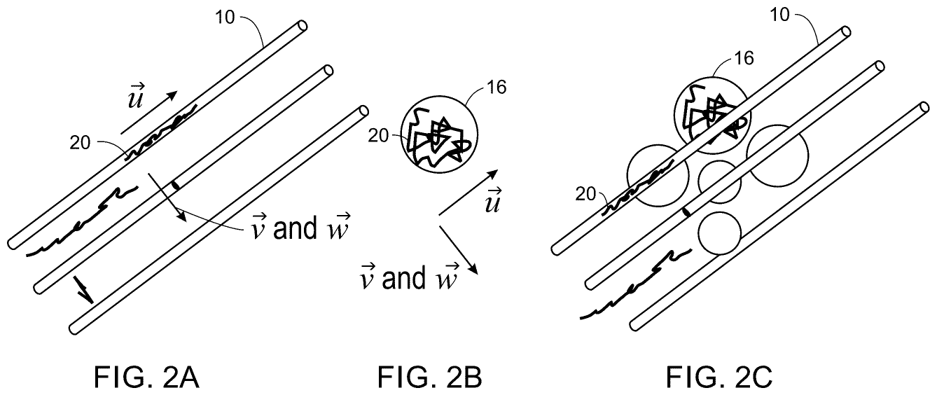

In the axon 10 in FIG. 2A, water molecular movement along the fiber direction, u, is free or hindered. Therefore, axial diffusivity (ADₐₓₒₙ, diffusion coefficient along the fiber direction) is relatively large. Water molecular movement perpendicular to the fiber direction \vec{v} and \vec{w} is restricted due to the cell membrane boundary. Therefore, the radial diffusivity (RDₐₓₒₙ, diffusion coefficient perpendicular to the fiber direction) is relatively small. The three eigenvalues for ADₐₓₒₙ=λ₁>>λ₂=λ₃=RDₐₓₒₙ. The FA for the axon (FAₐᵥₒₙ) is represented by the following:

$${FA}_{axon} = \sqrt{\frac{{( {AD}_{axon} - {RD}_{axon} )}^{2}}{{AD}_{axon}^{2} + 2 \cdot {RD}_{axon}^{2}}} \qquad ( 3 )$$

which is approximate to 1. Therefore, high FA has been used to indicate the presence of axons as the dominant tissue types in an imaging voxel of diffusion MRI.

However, water molecular movement outside axons (or in the extra-axonal space) complicates the AD, RD, FA in the image voxel of healthy white matter. The extra-axonal diffusion also presents anisotropy, with a larger ADₑₓ and smaller RDₑₓ. Due to a relatively larger space, RDₑₓ is relatively larger than RDₐₓₒₙ. Therefore, the measured diffusion parameters in the image voxel of healthy white matter have been characterized with high AD_{WM}, relatively low RD_{WM}, and a range of relatively high FA_{WM}.

In solid tumor of gliomas depicted in FIG. 2B, the diffusion process is close to isotropic. That is: ADₜᵤₘₒᵣ=RDₜᵤₘₒᵣ and FAₜᵤₘₒᵣ≈0. Therefore, low FA has been used to indicate the presence of solid tumor as the dominant tissue types in an imaging voxel of diffusion MRI. FIG. 2C depicts a mixture of axon fibers 10 and tumor cells 16. Lines 20 in FIGS. 2A-2C represent water molecular movements. Three-dimensional orthogonal diffusion directions in tissue are represented by u, v, and w.

In an image voxel with a mixture of axons 10, extra-axonal space, and tumor cells 16 depicted in FIG. 2C, for example, infiltrative tumor in gliomas, FAₘᵢₓ is reduced compared to FAₐₓₒₙ and increased compared to FAₜᵤₘₒᵣ. However, FAₘᵢₓ may still be in the range of FA_{WM}. That is, FAₜᵤₘₒᵣ<FAₘᵢₓ≈FA_{WM}<FAₐₓₒₙ. Therefore, these image voxels may be mistakenly identified as healthy white matter, due to the overlapping values of FAₘᵢₓ and FA_{WM}.

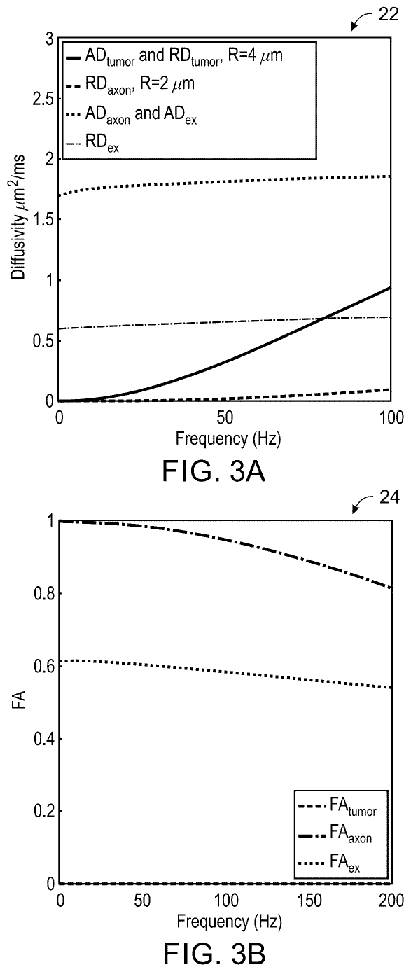

Previous studies reported no or weak diffusion time-dependency of ADₐₓₒₙ, RDₐₓₒₙ, ADₑₓ, and RDₑₓ in healthy human white matter as depicted in graph 22 of FIG. 3A. That is, these values do not vary significantly as a function of diffusion time or frequency. Therefore, FAₐₓₒₙ and FAₑₓ both present relatively high FA and week diffusion time-dependency as depicted in graph 24 of FIG. 3B. Graphs 22 and 24 depict a simulation of diffusivity and FA, respectively, as a function of diffusion time or frequency (x-axis) for different tissue types. As an example, a small radius of 2 micrometers is used in simulating axon diffusivities and a radius of 4 micrometers is used in simulating tumor diffusivities. Previous studies reported high diffusion time-dependency of ADₜᵤₘₒᵣ, Rdₜᵤₘₒᵣ as depicted in the graph 22 of FIG. 3A. Due to the isotropic diffusion, FAₜᵤₘₒᵣ presents low values and no diffusion time-dependency as depicted in the graph 24 of FIG. 3B.

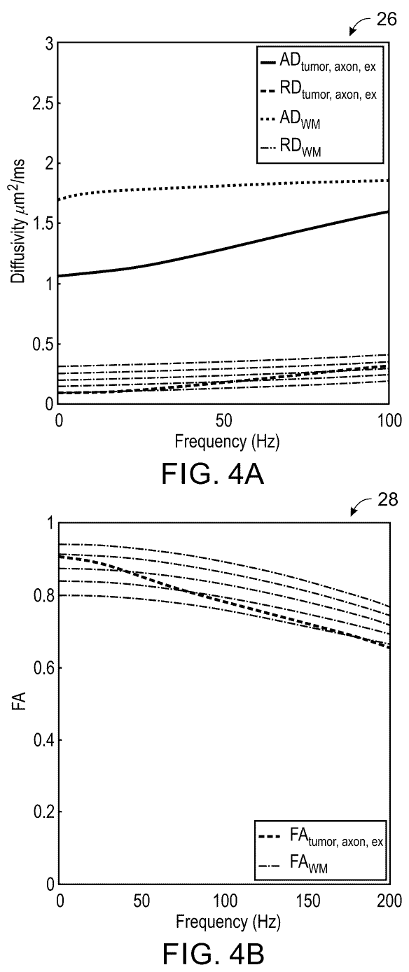

In white matter (simplified as a mixture of axons and extra-axonal space), both AD_{WM} and RD_{WM} present week diffusion time-dependency as depicted in graph 26 of FIG. 4A. Therefore, FA_{WM} presents weak diffusion time-dependency as depicted in graph 28 of FIG. 4B. Graphs 26 and 28 depict diffusivity and FA, respectively, of infiltrative tumor composed with tumor cells (20 percent volume fraction), axon (60 percent volume fraction), and extra-axonal space (20 percent volume fraction); and white matter composed with axons (80 percent to 40 percent volume fraction) and extra-axonal space (20 percent to 60 percent volume fraction).

Vasogenic edema can also be simplified as a mixture of axons and extra-axonal space filled with fluid. Since fluid has high diffusivity, RD_{VE} is increased compared to RD_{WM}, resulting in smaller FA_{VE} compared to FA_{WM}, AD_{VE}, RD_{VE}, and FA_{VE} all present week diffusion time-dependency.

In infiltrative tumor in gliomas (simplified as a mixture of axons, extra-axonal space, and tumor cells, AD_{tumor,axon,ex} presents high diffusion time-dependency and RD_{tumor,axon,ex} presents weak and medium diffusion time-dependency as depicted in the graph 26 of FIG. 4A. Therefore, FA_{tumor,axon,ex} presents high diffusion time-dependency) as depicted in the graph 28 of FIG. 4B.

Differentiating tumoral tissues from non-tumoral tissues is essential to the successful treatment of brain tumor patients. Treatment planning requires brain tissue classification to differentiate malignant solid tumor, mixtures of tumors and non-tumoral tissues (i.e., tumor infiltration), and benign lesions, such that the treatment can be optimized to preserve healthy brain tissues and brain function while maximally removing, damaging or killing tumoral tissues.

The present disclosure provides an MRI-based method for differentiating solid tumor, mixtures of tumors and non-tumoral tissues (i.e., tumor infiltration), and benign lesions. In particular, the MRI-based method identifies brain tissue types based on FA at long diffusion time (e.g., greater than 10 milliseconds) or low frequency (e.g., 20 hertz), and the relative change of FA and RD from long diffusion time to short diffusion time (i.e., less than 10 milliseconds) or high frequency (i.e., greater than 50 hertz). The disclosed technique helps the tumor board including radiologists, neurosurgeons, oncologists, and radiation oncologists to improve their planning for first-line treatment of brain tumors. It includes improved planning of surgical resection and better delineation of regions in radiation therapy. The disclosed techniques improves treatment of brain tumors including surgery, radiation therapy, chemotherapy, and other therapies. Furthermore, the disclosed techniques help the tumor board to increase the confidence for early identification of tumor recurrence after first-line treatment such that patients can timely undergo second-line treatment to improve treatment efficacy and survival rate.

In the following disclosure, the disclosed techniques are described with regard to the brain. However, the disclosed techniques may be also utilized with other tissues.

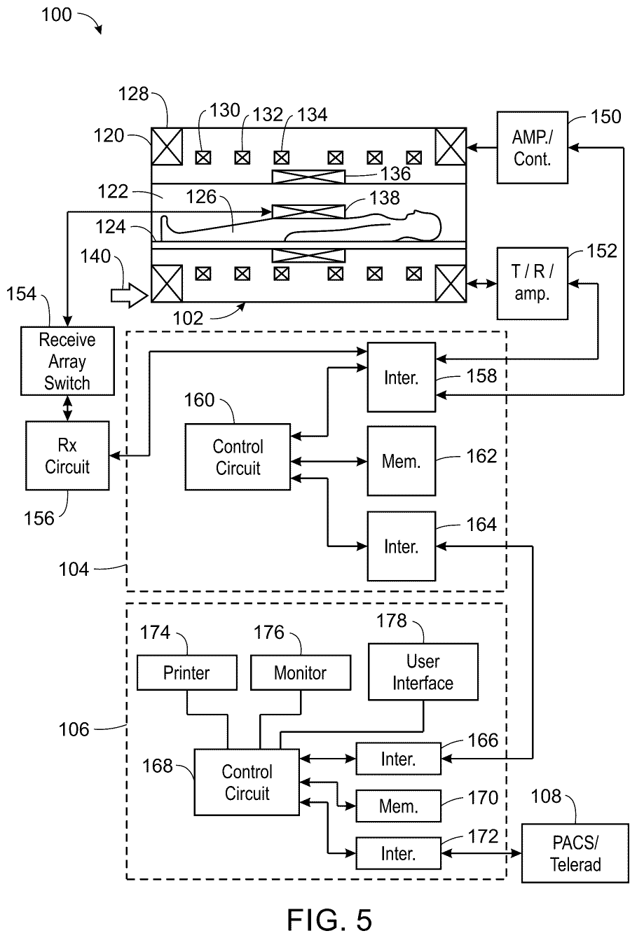

With the preceding in mind, FIG. 5 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally in parallel to the longest axis of the bore 122. A series of gradient coils 130, 132, and 134 permit controlled magnetic gradient fields to be generated for positional encoding of certain gyromagnetic nuclei within the patient 126 during examination sequences. A radio frequency (RF) transmit coil 136 is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the RF signals that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, B₀. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF transmit coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the RF receiving coils 138 are connected to switch 154, which is capable of switching the RF receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the RF receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute instructions that, when executed by a processor, are configured to perform reconstruction of acquired data as described below. In certain embodiments, the memory circuit 170 may store instructions for classifying brain tissue. In certain embodiments, the techniques disclosed herein may occur on a separate computing device having processing circuitry and memory circuitry.

A processing component (e.g., a microprocessor or processing circuitry) and a memory of the magnetic resonance imaging system 100, such as may be present in scanner control circuitry 104 and/or system control circuitry 106, may be used to execute stored software code, instructions, or routines for acquiring and processing the MR data. The term “code” or “software code” used herein refers to any instructions or set of instructions that control the magnetic resonance imaging system 100. The code or software code may exist in a computer-executable form, such as machine code, which is the set of instructions and data directly executed by the processing component of the scanner control circuitry 104 and/or system control circuitry 106, human-understandable form, such as source code, which may be compiled in order to be executed by the processing component of the scanner control circuitry 104 and/or system control circuitry 106, or an intermediate form, such as object code, which is produced by a compiler. In some embodiments, the magnetic resonance imaging system 100 may include a plurality of controllers.

As an example, the memory may store processor-executable software code or instructions (e.g., firmware or software), which are tangibly stored on a non-transitory computer readable medium. Additionally or alternatively, the memory may store data. As an example, the memory may include a volatile memory, such as random-access memory (RAM), and/or a nonvolatile memory, such as read-only memory (ROM), flash memory, a hard drive, or any other suitable optical, magnetic, or solid-state storage medium, or a combination thereof. Furthermore, processing component may include multiple microprocessors, one or more “general-purpose” microprocessors, one or more special-purpose microprocessors, and/or one or more application specific integrated circuits (ASICS), or some combination thereof. For example, the processing component may include one or more reduced instruction set (RISC) or complex instruction set (CISC) processors. The processing component may include multiple processors, and/or the memory may include multiple memory devices.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

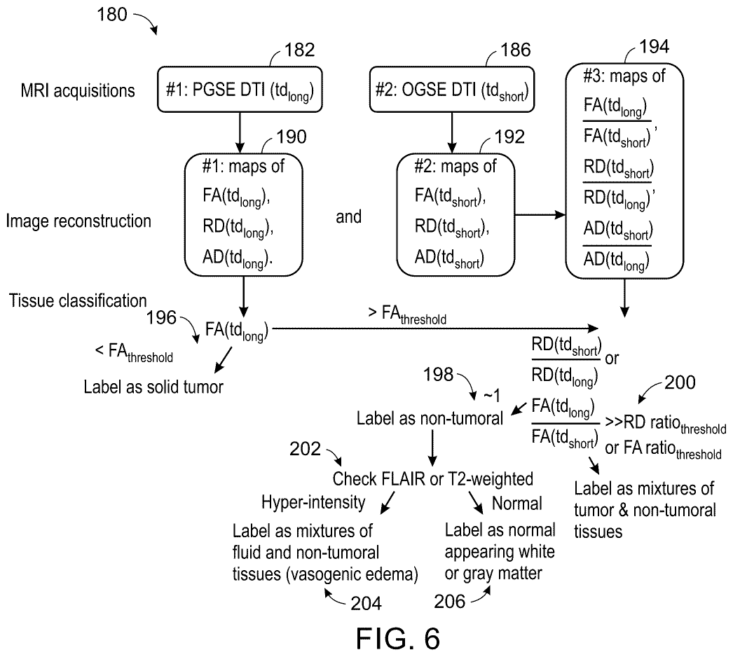

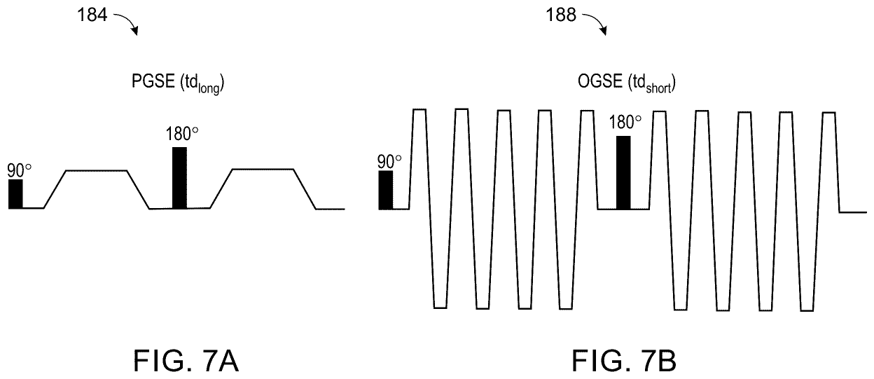

FIG. 6 depicts a schematic diagram of a workflow 180 for MRI-based brain tissue classification. The workflow 180 includes performing DTI MRI acquisitions with two different diffusion times. In particular, the workflow 180 includes performing a DTI MRI acquisition of a brain of a subject (e.g., patient) utilizing a pulsed gradient spin echo (PGSE) diffusion encoding which is used to acquire images at a long diffusion time (td_{long}, e.g., with a diffusion time greater than 10 milliseconds or low frequency of 20 hertz) as indicated by reference numeral 182. FIG. 7A depicts a pulse diagram 184 of a pulse sequence using PGSE diffusion encoding. The workflow 180 also includes performing a DTI MRI acquisition of the brain of the subject utilizing an oscillating gradient spin echo (OGSE) diffusion encoding to acquired images at a short diffusion time (tdₛₕₒᵣₜ, e.g., with a diffusion time less than 10 milliseconds or a high frequency of greater than 20 hertz) as indicated by reference numeral 186. FIG. 7B depicts a pulse diagram 188 of a pulse sequence using OGSE diffusion encoding. In each of the acquisitions, multiple diffusion encoding directions are acquired at a single b-value or multiple b-values.

The workflow 180 also includes performing image reconstruction. In particular, the workflow 180 includes calculating FA, RD, and AD for the PGSE DTI dataset via reconstructing respective maps FA (td_{long}), RD (td_{long}), and AD (td_{long}) as indicated by reference numeral 190. The workflow 180 also includes calculating FA, RD, and AD for the OGSE DTI dataset via reconstructing respective maps FA (tdₛₕₒᵣₜ), RD (tdₛₕₒᵣₜ), and AD(tdₛₕₒᵣₜ) as indicated by reference numeral 192. Then, the workflow 182 includes reconstructing (i.e., generating) maps of

$$\frac{FA ( {td}_{long} )}{FA ( {td}_{short} )} , \frac{RD ( {td}_{short} )}{RD ( {td}_{long} )} , and \frac{AD ( {td}_{short} )}{AD ( {td}_{long} )}$$

as indicated by reference numeral 194.

The workflow 180 further includes performing tissue classification of the brain of the subject. Prior to classification (via a separate MRI acquisition of the brain of the subject), enhancing lesions and non-enhancing lesions are identified from clinical standard T1-weighted contrast-enhanced imaging and T2-weighted Flair or T2-weighted periodically rotated overlapping parallel lines with enhanced reconstruction (PROPELLER). A lesion mask is generated from these. A FA threshold is defined and used to compare to the respective FA value (from FA(td_{long})) in each voxel inside the lesion mask as indicated by reference numeral. When the respective FA value of the voxel inside the lesion mask is equal to or lower than the FA threshold, the tissue (i.e., the voxel) is labeled as solid tumor as indicated by reference numeral 196. When the respective FA value of the voxel inside the lesion mask is greater than the FA threshold, either a RD ratio_{threshold} or a FA ratio_{threshold} is defined. Then, either the ratio value

$$( \frac{RD ( {td}_{short} )}{RD ( {td}_{long} )}$$

### Value) or the Ratio Value

$$( \frac{FA ( {td}_{long} )}{FA ( {td}_{short} )}$$

value) of the respective voxel in the lesion mask is compared to the RD ratio_{threshold} or the FA ratio_{threshold}, respectively. When either the ratio value

$$( \frac{RD ( {td}_{short} )}{RD ( {td}_{long} )}$$

value) or the ratio value

$$( \frac{FA ( {td}_{long} )}{FA ( {td}_{short} )}$$

value) of the respective voxel in the lesion mask is equal to or less than the RD ratio_{threshold} or the FA ratio_{threshold} (e.g., approximately 1), respectively, then the tissue (i.e., the respective voxel) is labeled as non-tumoral as indicated by reference numeral 198. When either the ratio value

$$( \frac{RD ( {td}_{short} )}{RD ( {td}_{long} )}$$

value) or the ratio value

$$( \frac{FA ( {td}_{long} )}{FA ( {td}_{short} )}$$

value) of the respective voxel in the lesion mask is greater than the RD ratio_{threshold} or the FA ratio_{threshold} (e.g., approximately 1), respectively, then the tissue (i.e., the respective voxel) is labeled as a mixture of tumor and non-tumoral tissues as indicated by reference numeral 200. To further classify the voxels in the lesion mask that are classified as non-tumoral, the corresponding voxels are further examined in FLAIR or T2-weighted images (obtained via a separate acquisition of the clinical routine brain MRI) as indicated by reference numeral 202. Voxels in the lesion mask classified as non-tumoral that appear as hyper-intensive in the FLAIR or T2-weighted images are labeled as mixtures as fluid and non-tumoral tissues (vasogenic edema) as indicated by reference numeral 204. Voxels in the lesion mask classified as non-tumoral that appear as normal in the FLAIR or T2-weighted images are labeled as normal appearing white matter or gray matter as indicated by reference numeral 206.

In each image voxel inside the lesion mask, a tissue label is assigned based on the values of FA (from FA (td_{long}),

$$\frac{FA ( {td}_{long} )}{FA ( {td}_{short} )} , and \frac{RD ( {td}_{short} )}{RD ( {td}_{long} )} ) .$$

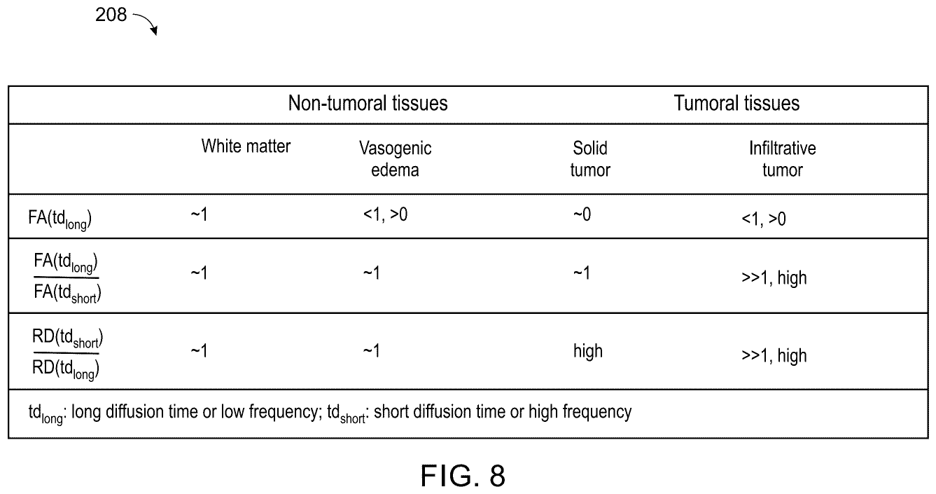

For example, 1 equals solid tumor, 2 equals mixtures of tumor and non-tumoral tissue, and 3 equals non-tumoral tissue or benign tissue. Table 208 in FIG. 8 summarizes time-dependent diffusivity and fractional anisotropy of different tissue types.

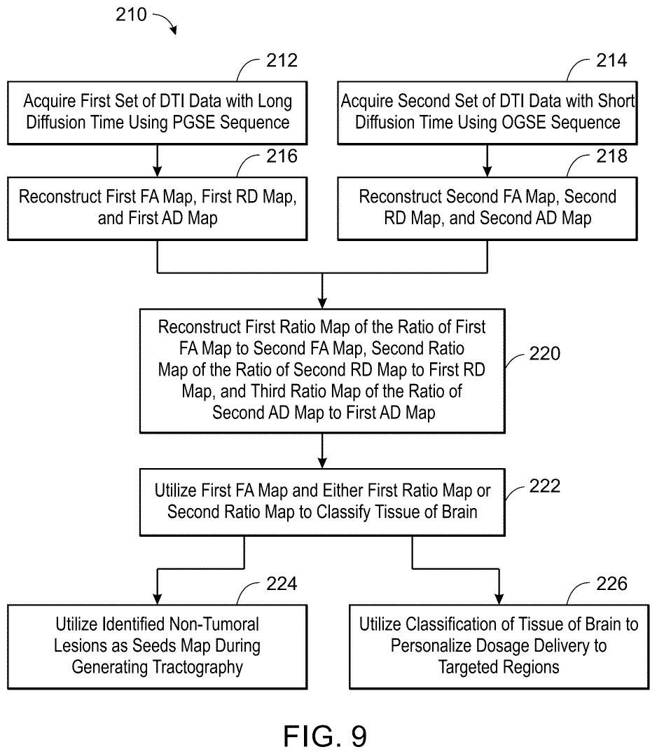

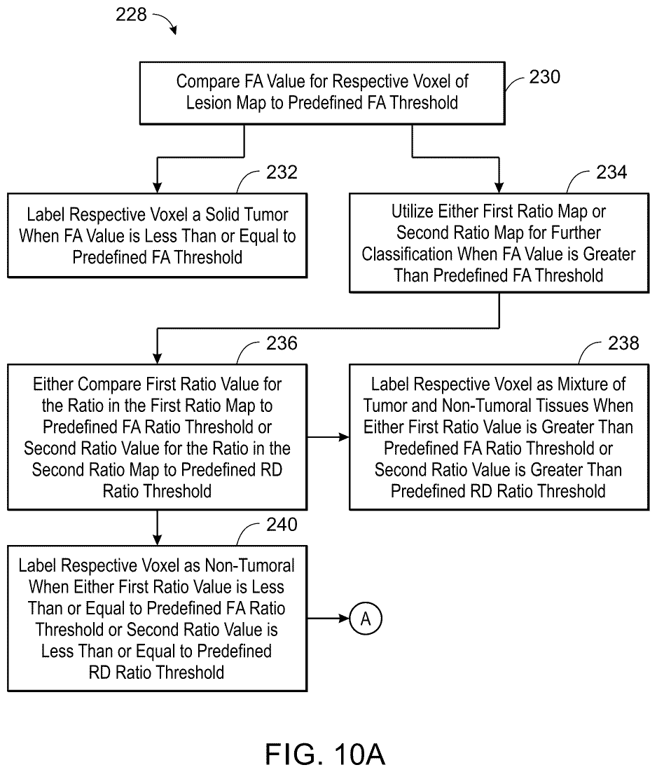

FIG. 9 illustrates a flow diagram of a method 210 for MRI-based brain tissue classification. One or more steps of the method 210 may be performed by processing circuitry of the magnetic resonance imaging system 100 in FIG. 5 or a remote computing device. One or more of the steps of the method 210 may be performed simultaneously and/or in a different order from that depicted in FIG. 9.

The method 210 includes acquiring a first set of diffusion tensor imaging data having a long diffusion time (td_{long}) of a brain of a subject with a magnetic resonance imaging (MRI) scanner utilizing a pulsed gradient spin echo pulse sequence (block 212). The method 210 also includes acquiring a second set of diffusion tensor imaging data having a short diffusion time (tdₛₕₒᵣₜ) that is shorter than the long diffusion time of the brain of the subject with the MRI scanner utilizing an oscillating gradient spin echo pulse sequence (block 214). The method 210 further includes reconstructing a first fractional anisotropy map (FA (td_{long})), a first radial diffusivity map (RD (td_{long})), and a first axial diffusivity map (AD (td_{long})) from the first set of diffusion tensor imaging data having the long diffusion time (block 216). The method 210 even further includes reconstructing a second fractional anisotropy map (FA (tdₛₕₒᵣₜ)), a second radial diffusivity map (RD (tdₛₕₒᵣₜ)), and a second axial diffusivity map (AD (tdₛₕₒᵣₜ)) from the second set of diffusion tensor imaging data having the short diffusion time (block 218). The method 210 also includes reconstructing a first ratio map

$$( \frac{FA ( {td}_{long} )}{FA ( {td}_{short} )}$$

map) based on the ratio of the first fractional anisotropy map to the second fractional anisotropy map, a second ratio map

$$( \frac{RD ( {td}_{short} )}{RD ( {td}_{long} )}$$

map) based on the ratio of the second radial diffusivity map to the first radial diffusivity map, and a third ratio map

$$( \frac{AD ( {td}_{short} )}{AD ( {td}_{long} )}$$

map) based on the ratio of the second axial diffusivity map to the first axial diffusivity map (block 220). The method 210 further includes utilizing the first fractional anisotropy map and either the first ratio map or the second ratio map to classify tissue of the brain (block 222).

In certain embodiments, the method 210 includes utilizing non-tumoral lesions identified in the classification of the tissue of the brain as a seeds map during generating tractography of the brain of the subject (block 224). In certain embodiments, the method 210 includes utilizing the classification of the tissue of the brain to personalize dosage delivery to targeted regions for radiation therapy treatment of the brain of the subject (block 226).

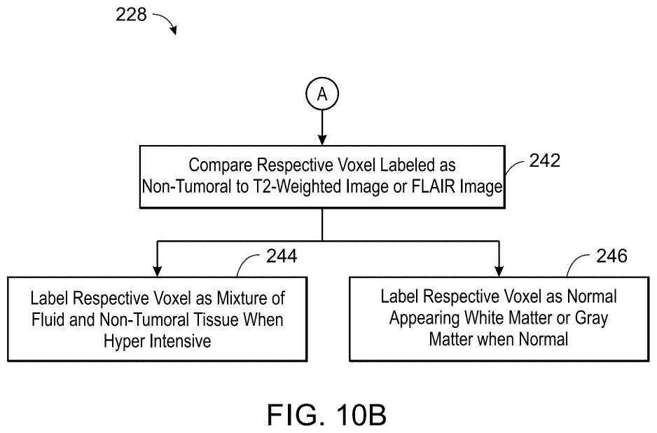

FIGS. 10A and 10B illustrate a flow diagram of a method 228 for the tissue classification step 222 of the method 210 in FIG. 9. One or more steps of the method 228 may be performed by processing circuitry of the magnetic resonance imaging system 100 in FIG. 5 or a remote computing device. One or more of the steps of the method 228 may be performed simultaneously and/or in a different order from that depicted in FIGS. 10A and 10B. The method 228 applies to each voxel within a lesion map of an MRI image of the brain of the subject, wherein the lesion map identifies all lesion types such as solid tumor, mixture of tumor and non-tumoral tissues, mixture of fluid and non-tumoral tissues (vasogenic edema), and normal appearing white matter and gray matter.

The method 228 includes comparing a fractional anisotropy value for a respective voxel of the lesion map in the first fractional anisotropy map to a predefined fractional anisotropy threshold (block 230). The method 228 also includes labeling the respective voxel of the lesion map as a solid tumor when the fractional anisotropy value is less than or equal to the predefined fractional anisotropy threshold (block 232). The method 228 further includes utilizing either the first ratio map or the second ratio map for further classification of the tissue of the brain when the fractional anisotropy value is greater than the predefined fractional anisotropy threshold (block 234).

When utilizing either the first ratio map or the second ratio map for further classification of the tissue of the brain when the fractional anisotropy value is greater than the predefined fractional anisotropy threshold, the method 228 includes either comparing a first ratio value for the ratio for the respective voxel in the lesion mask in the first ratio map to a predefined fractional anisotropy ratio threshold (FA ratio_{threshold}) or a second ratio value for the respective voxel in the lesion mask in the second ratio map to a predefined radial diffusivity ratio threshold (RD ratio_{threshold}) (block 236). The method 228 also includes labeling the respective voxel of the lesion map as a mixture of tumor and non-tumoral tissue when either the first ratio value is greater than the second predefined fractional anisotropy ratio threshold or the second ratio value is greater than the predefined radial diffusivity ratio threshold (block 238). The method 228 further includes labeling the respective voxel of the lesion map as non-tumoral when either the first ratio value is less than or equal to the second predefined fractional anisotropy ratio threshold or the second ratio value is less than or equal to the predefined radial diffusivity ratio threshold (block 240).

The method 228 includes comparing the respective voxel labeled as non-tumoral to a T2-weighted image or fluid-attenuated inversion recovery (FLAIR) image of the brain of the subject (block 242). The method 228 also includes further labeling the respective voxel labeled as non-tumoral as a mixture of fluid and non-tumoral tissue when the comparison determines the respective voxel is hyper intensive in T2-weighted image or FLAIR image (block 244). The method 228 includes further labeling the respective voxel labeled as non-tumoral as normal appearing white matter or gray matter when the comparison determines the respective voxel is normal in T2-weighted image or FLAIR image (block 246).

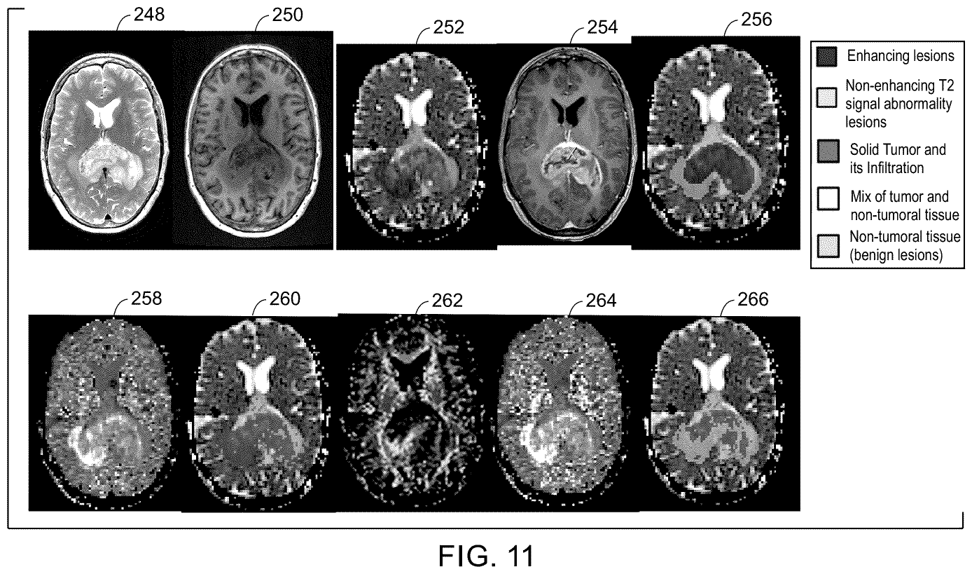

FIG. 11 depicts images of a brain of a subject (e.g., diagnosed with glioblastoma) comparing tissue classification between the disclosed technique and current techniques. Image 248 is a T2 PROPELLER image. Image 250 is a T1 magnetization-prepared rapid gradient echo (MPRAGE) image. Image 252 is an apparent diffusion coefficient (ADC) image at 0 hertz that is acquired using PGSE diffusion encoding. Image 254 is a contrast-enhanced image. Image 256 is a lesion mask. Images 248-256 represent image acquired and tissue labeling in current clinical practice. In current clinical practice, only labels tissues as enhancing lesions (likely solid tumor) and non-enhancing abnormal lesions. Non-enhancing abnormal tissues could be either tumoral or non-tumoral benign lesions. Image 258 is a

$$\frac{ADC ( {td}_{short} )}{ADC ( {td}_{long} )}$$

image. Image 260 is a tissue classification image. Image 260 represents labeling utilizing a state-of-the-art technique with image 258. The current state-of-the-art techniques cannot differentiate solid tumor from infiltrative tumor (thus, creating uncertainty whether tissues should be resected or radiated). Image 262 is a color FA image at 0 hertz that is acquired using PGSE diffusion encoding. Image is a

$$\frac{RD ( {td}_{short} )}{RD ( {td}_{long} )}$$

image. Image 266 is a tissue classification image. Images 262-266 are derived from utilizing the disclosed technique described in FIGS. 6, 9, 10A, and 10B. As depicted in image 266, each image voxel inside the lesion mask is assigned a tissue label based on the values of FA (from FA (td_{long}),

$$\frac{FA ( {td}_{long} )}{FA ( {td}_{short} )} , and \frac{RD ( {td}_{short} )}{RD ( {td}_{long} )} ) .$$

For example, the images voxels in the lesion mask in image 266 are labeled as solid tumor, mixtures of tumor and non-tumoral tissue, and non-tumoral tissue or benign tissue.

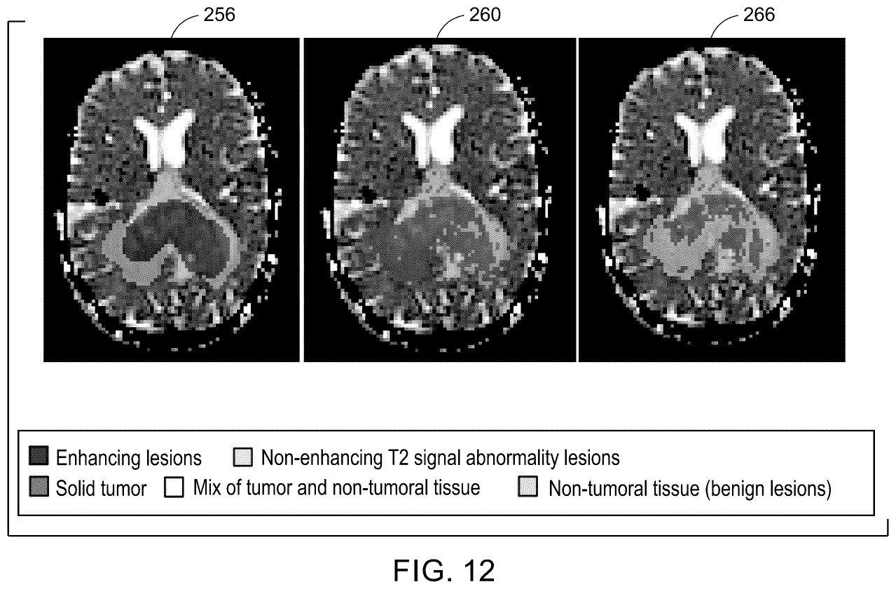

FIG. 12 depicts images for illustrating treatment planning using different maps (from the different tissue classification techniques in FIG. 11) for guidance. FIG. 12 includes image 256 (lesion mask image), image 260 (tissue classification image using state-of-the-art techniques), and image 266 (tissue classification image from using disclosed technique) from FIG. 11. The problem with utilizing image 256 in treatment planning is that it is not clear if the non-enhancing T2 signal abnormality regions are tumoral or non-tumor tissues. Therefore, treatment regions can be overly large or underestimated. The problem with utilizing image 260 in treatment planning is that it is not able to differentiate solid tumor from infiltrative tumor, which leaves the fiber tracks undetermined and treatment procedures potentially compromised. The advantage with utilizing image 266 is that the tissue classification provides guidance for personalized treatment planning.

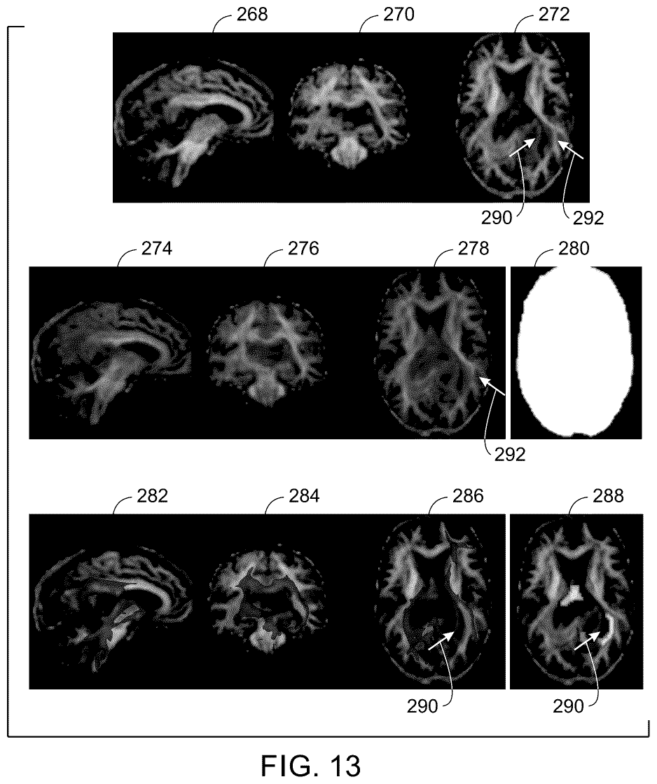

FIG. 13 illustrates the utilization of brain tissue classification utilizing the disclosed technique in tractography. Images 268, 270, and 272 are FA images of a brain of a subject from DTI. Images 268, 270, and 272 are sagittal, coronal, and axial views, respectively. Images 274, 276, and 278 depict tracts on the images 268, 270, and 272 utilizing whole brain seeds map 280 (which is currently used) in generating the tracts based on the FA values. Images 282, 284, and 286 depict tracts on the images 268, 270, and 272 using a non-tumoral lesions seeds map 288 (derived from the disclosed technique in FIGS. 6, 9, 10A, and 10B. The non-tumoral lesions were identified using the disclosed technique. Arrows 290 indicate low FA in a potential vasogenic region. Arrows 292 indicate where tracts do not go through vasogenic edema regions which occurs in image 278. In image 286, the generated tracts go through vasogenic edema regions as expected. The information guides the neurosurgeons not to resect these regions, and the radiation oncologists not to radiate these regions, to avoid damaging normal tracts and the function of the patient brain.

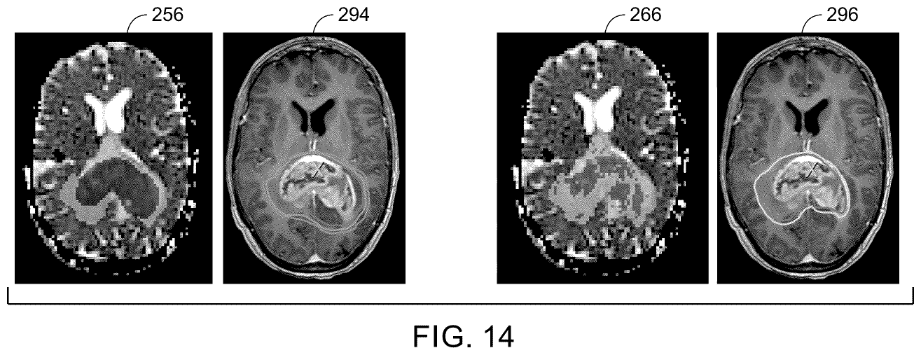

FIG. 14 illustrates the utilization of brain tissue classification utilizing the disclosed technique in dosage delivery in radiation therapy. Image 256 (lesion mask image) and image 266 (tissue classification image from using disclosed technique) are from FIG. 11. Image 294 indicates the dosage delivery regions in radiation therapy relying on the current standard enhancing lesions and non-enhancing abnormal regions (i.e., utilizing image 256). In particular, a high dosage is applied to the enhancing regions and a medium or high dosage to non-enhancing T2 signal abnormal lesions, which may undertreat infiltrative tumor or overtreat benign lesion due to the lack of accurate tissue classification.

Utilizing image 266 personalizes treatment to targeting regions (i.e., providing personalized dosage delivery regions) in solid tumor and its infiltration as indicate in image 296. The information in image 266 may guide radiation oncologists to increase dosage to the solid tumor and non-enhancing infiltration tumor to maximally damage or kill tumor tissues, and reduce the dosage to benign lesions to avoid damaging normal tracts and the function of the patient brain. Similarly, the disclosed tissue classification technique may be utilized to personalize treatment for therapeutic deliveries (e.g., drug delivery) via focused ultrasound or other methods.

Technical effects of the disclosed subject matter include providing treatment planning on brain tumor patients that will be more accurate and personalized. Technical effects of the disclosed subject matter include enabling the biopsy or resection to be more targeted to solid tumor. Technical effects of the disclosed subject matter include lowering the risk of damaging nerves. Technical effects of the disclosed subject matter include enabling the function of tissues that are not tumor or its infiltration to more likely be preserved in radiation therapy and/or therapeutic delivery. Technical effects of the disclosed subject matter include increasing the success of treatment and patient quality of life.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform] ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for classifying brain tissue, comprising:
acquiring, via a processing system comprising one or more processors, a first set of diffusion tensor imaging data having a long diffusion time of a brain of a subject with a magnetic resonance imaging (MRI) scanner utilizing a pulsed gradient spin echo pulse sequence;
acquiring, via the processing system, a second set of diffusion tensor imaging data having a short diffusion time that is shorter than the long diffusion time of the brain of the subject with the MRI scanner utilizing an oscillating gradient spin echo pulse sequence;
reconstructing, via the processing system, a first fractional anisotropy map, a first radial diffusivity map, and a first axial diffusivity map from the first set of diffusion tensor imaging data having the long diffusion time;
reconstructing, via the processing system, a second fractional anisotropy map, a second radial diffusivity map, and a second axial diffusivity map from the second set of diffusion tensor imaging data having the short diffusion time;
reconstructing, via the processing system, a first ratio map based on the ratio of the first fractional anisotropy map to the second fractional anisotropy map, a second ratio map based on the ratio of the second radial diffusivity map to the first radial diffusivity map, and a third ratio map based on the ratio of the second axial diffusivity map to the first axial diffusivity map; and
utilizing, via the processing system, the first fractional anisotropy map and either the first ratio map or the second ratio map to classify tissue of the brain.

2. The computer-implemented method of claim 1, wherein utilizing the first fractional anisotropy map and either the first ratio map or the second ratio map to classify the tissue of the brain further comprises:
for each voxel within a lesion map of a contrast MRI image of the brain of the subject, wherein the lesion map identifies enhancing lesions and/or non-enhancing lesions:
comparing, via the processing system, a fractional anisotropy value for a respective voxel of the lesion map in the first fractional anisotropy map to a predefined fractional anisotropy threshold;
labeling, via the processing system, the respective voxel of the lesion map as a solid tumor when the fractional anisotropy value is less than or equal to the predefined fractional anisotropy threshold; and
utilizing, via the processing system, either the first ratio map or the second ratio map for further classification of the tissue of the brain when the fractional anisotropy value is greater than the predefined fractional anisotropy threshold.

3. The computer-implemented method of claim 2, wherein utilizing either the first ratio map or the second ratio map for further classification of the tissue of the brain when the fractional anisotropy value is greater than the predefined fractional anisotropy threshold further comprises:
either comparing, via the processing system, a first ratio value for the first ratio for the respective voxel in the lesion mask in the first ratio map to the predefined fractional anisotropy ratio threshold or a second ratio value for the second ratio for the respective voxel in the lesion mask in the second ratio map to a predefined radial diffusivity ratio threshold; and
labeling, via the processing system, the respective voxel of the lesion map as a mixture of tumor and non-tumoral tissue when either the first ratio value is greater than the predefined fractional anisotropy ratio threshold or the second ratio value is greater than the predefined radial diffusivity ratio threshold.

4. The computer-implemented method of claim 3, wherein utilizing either the first ratio map or the second ratio map for further classification of the tissue of the brain when the fractional anisotropy value is greater than the predefined fractional anisotropy threshold further comprises:
labeling, via the processing system, the respective voxel of the lesion map as non-tumoral when either the first ratio value is less than or equal to the predefined fractional anisotropy ratio threshold or the second ratio value is less than or equal to the predefined radial diffusivity ratio threshold.

5. The computer-implemented method of claim 4, further comprising:
comparing, via the processing system, the respective voxel labeled as non-tumoral to a T2-weighted image or fluid-attenuated inversion recovery image of the brain of the subject;
further labeling, via the processing system, the respective voxel labeled as non-tumoral as a mixture of fluid and non-tumoral tissue when the comparison determines the respective voxel is hyper intensive; and
further labeling, via the processing system, the respective voxel labeled as non-tumoral as normal appearing white matter or gray matter when the comparison determines the respective voxel is normal.

6. The computer-implemented method of claim 1, further comprising utilizing, via the processing system, non-tumoral lesions identified in the classification of the tissue of the brain as a seeds map during generating tractography of the brain of the subject.

7. The computer-implemented method of claim 1, further comprising utilizing, via the processing system, the classification of the tissue of the brain to personalize dosage delivery to targeted regions for radiation therapy treatment of the brain of the subject.

8. A system for classifying brain tissue, comprising:
a memory encoding processor-executable routines; and
a processing system comprising one or more processors and configured to access the memory and to execute the processor-executable routines, wherein the processor-executable routines, when executed by the processing system, cause the processing system to:
acquire a first set of diffusion tensor imaging data having a long diffusion time of a brain of a subject with a magnetic resonance imaging (MRI) scanner utilizing a pulsed gradient spin echo pulse sequence;
acquire a second set of diffusion tensor imaging data having a short diffusion time that is shorter than the short diffusion time of the brain of the subject with the MRI scanner utilizing an oscillating gradient spin echo pulse sequence;
reconstruct a first fractional anisotropy map, a first radial diffusivity map, and a first axial diffusivity map from the first set of diffusion tensor imaging data having the long diffusion time;
reconstruct a second fractional anisotropy map, a second radial diffusivity map, and a second axial diffusivity map from the second set of diffusion tensor imaging data having the short diffusion time;
reconstruct a first ratio map based on the ratio of the first fractional anisotropy map to the second fractional anisotropy map, a second ratio map based on the ratio of the second radial diffusivity map to the first radial diffusivity map, and a third ratio map based on the ratio of the second axial diffusivity map to the first axial diffusivity map; and
utilize the first fractional anisotropy map and either the first ratio map or the second ratio map to classify tissue of the brain.

9. The system of claim 8, wherein for utilizing the first fractional anisotropy map and either the first ratio map or the second ratio map to classify the tissue of the brain, the processor-executable routines, when executed by the processing system, further cause the processing system to:
for each voxel within a lesion map of a post-contrast enhancing MRI image of the brain of the subject, wherein the lesion map identifies enhancing lesions and/or non-enhancing lesions:
compare a fractional anisotropy value for a respective voxel of the lesion map in the first fractional anisotropy map to a predefined fractional anisotropy threshold;
label the respective voxel of the lesion map as a solid tumor when the fractional anisotropy value is less than or equal to the predefined fractional anisotropy threshold; and
utilize the first ratio map or the second ratio map for further classification of the tissue of the brain when the fractional anisotropy value is greater than the predefined fractional anisotropy threshold.

10. The system of claim 9, wherein for utilizing either the first ratio map or the second ratio map for further classification of the tissue of the brain when the fractional anisotropy value is greater than the predefined fractional anisotropy threshold, the processor-executable routines, when executed by the processing system, further cause the processing system to:
either compare a first ratio value for the first ratio for the respective voxel in the lesion mask in the first ratio map to a predefined fractional anisotropy ratio threshold or a second ratio value for the second ratio for the respective voxel in the lesion mask in the second ratio map to a predefined radial diffusivity ratio threshold; and
label the respective voxel of the lesion map as a mixture of tumor and non-tumoral tissue when either the first ratio value is greater than the predefined fractional anisotropy ratio threshold or the second ratio value is greater than the predefined radial diffusivity ratio threshold.

11. The system of claim 10, wherein for utilizing either the first ratio map or the second ratio map for further classification of the tissue of the brain when the fractional anisotropy value is greater than the predefined fractional anisotropy threshold, the processor-executable routines, when executed by the processing system, further cause the processing system to:
label the respective voxel of the lesion map as non-tumoral when either the first ratio value is less than or equal to the predefined fractional anisotropy ratio threshold or the second ratio value is less than or equal to the predefined radial diffusivity ratio threshold.

12. The system of claim 11, wherein the processor-executable routines, when executed by the processing system, further cause the processing system to:
compare the respective voxel labeled as non-tumoral to a T2-weighted image or fluid-attenuated inversion recovery (FLAIR) image of the brain of the subject;
further label the respective voxel labeled as non-tumoral as a mixture of fluid and non-tumoral tissue when the comparison determines the respective voxel is hyper intensive in T2-weighted image or FLAIR image; and
further label the respective voxel labeled as non-tumoral as normal appearing white matter or gray matter when the comparison determines the respective voxel is normal in T2-weighted image or FLAIR image.

13. The system of claim 8, wherein the processor-executable routines, when executed by the processing system, further cause the processing system to utilize non-tumoral lesions identified in the classification of the tissue of the brain as a seeds map during generating tractography of the brain of the subject.

14. The system of claim 8, wherein the processor-executable routines, when executed by the processing system, further cause the processing system to utilize the classification of the tissue of the brain to personalize dosage delivery to targeted regions for radiation therapy treatment of the brain of the subject.

15. A non-transitory computer-readable medium, the non-transitory computer-readable medium comprising processor-executable code that when executed by a processing system comprising one or more processors, causes the processing system to:
acquire a first set of diffusion tensor imaging data having a long diffusion time of a brain of a subject with a magnetic resonance imaging (MRI) scanner utilizing a pulsed gradient spin echo pulse sequence;
acquire a second set of diffusion tensor imaging data having a short diffusion time that is shorter than the long diffusion time of the brain of the subject with the MRI scanner utilizing an oscillating gradient spin echo pulse sequence;
reconstruct a first fractional anisotropy map, a first radial diffusivity map, and a first axial diffusivity map from the first set of diffusion tensor imaging data having the long diffusion time;
reconstruct a second fractional anisotropy map, a second radial diffusivity map, and a second axial diffusivity map from the second set of diffusion tensor imaging data having the short diffusion time;
reconstruct a first ratio map based on the ratio of the first fractional anisotropy map to the second fractional anisotropy map, a second ratio map based on the ratio of the second radial diffusivity map to the first radial diffusivity map, and a third ratio map based on the ratio of the second axial diffusivity map to the first axial diffusivity map; and
utilize the first fractional anisotropy map and either the first ratio map or the second ratio map to classify tissue of the brain.

16. The non-transitory computer-readable medium of claim 15, wherein for utilizing the first fractional anisotropy map and either the first ratio map or the second ratio map to classify the tissue of the brain, the processor-executable code, when executed by the processing system, further causes the processing system to:
for each voxel within a lesion map of a contrast MRI image of the brain of the subject, wherein the lesion map identifies enhancing lesions and/or non-enhancing lesions:
compare a fractional anisotropy value for a respective voxel of the lesion map in the first fractional anisotropy map to a predefined fractional anisotropy threshold;
label the respective voxel of the lesion map as a solid tumor when the fractional anisotropy value is less than or equal to the predefined fractional anisotropy threshold; and
utilize the first ratio map or the second ratio map for further classification of the tissue of the brain when the fractional anisotropy value is greater than the predefined fractional anisotropy threshold.

17. The non-transitory computer-readable medium of claim 16, wherein for utilizing either the first ratio map or the second ratio map for further classification of the tissue of the brain when the fractional anisotropy value is greater than the predefined fractional anisotropy threshold, the processor-executable code, when executed by the processing system, further causes the processing system to:
either compare a first ratio value for the first ratio for the respective voxel in the lesion mask in the first ratio map to a predefined fractional anisotropy ratio threshold or a second ratio value for the second ratio for the respective voxel in the lesion mask in the second ratio map to a predefined radial diffusivity ratio threshold; and
label the respective voxel of the lesion map as a mixture of tumor and non-tumoral tissue when either the first ratio value is greater than the predefined anisotropy ratio threshold or the second ratio value is greater than the predefined radial diffusivity ratio threshold.

18. The non-transitory computer-readable medium of claim 17, wherein for utilizing either the first ratio map or the second ratio map for further classification of the tissue of the brain when the fractional anisotropy value is greater than the predefined fractional anisotropy threshold, the processor-executable code, when executed by the processing system, further causes the processing system to:
label the respective voxel of the lesion map as non-tumoral when either the first ratio value is less than or equal to the predefined anisotropy ratio threshold or the second ratio value is less than or equal to the predefined radial diffusivity ratio threshold.

19. The non-transitory computer-readable medium of claim 18, wherein the processor-executable code, when executed by the processing system, further causes the processing system to:
compare the respective voxel labeled as non-tumoral to a T2-weighted image or fluid-attenuated inversion recovery (FLAIR) image of the brain of the subject;
further label the respective voxel labeled as non-tumoral as a mixture of fluid and non-tumoral tissue when the comparison determines the respective voxel is hyper intensive in T2-weighted image or FLAIR image; and
further label the respective voxel labeled as non-tumoral as normal appearing white matter or gray matter when the comparison determines the respective voxel is normal in T2-weighted image or FLAIR image.

20. The non-transitory computer-readable medium of claim 15, wherein the processor-executable code, when executed by the processing system, further causes the processing system to utilize non-tumoral lesions identified in the classification of the tissue of the brain as a seeds map during generating tractography of the brain of the subject.
